(19) United States
(12) Patent Application Publication
(10) Pub. No.: US 2002/0051995 A1
(43) Pub. Date: May 2, 2002

(54) Stacked arrays

(76) Inventor: Rajan Kumar, Robbinsville, NJ (US)

Correspondence Address: Rajan Kumar, 18 Buford Road, Robbinsville, NJ, 08691, US

(21) Appl. No.: 10/017,536

(22) Filed: Oct. 29, 2001

Related U.S. Application Data
(60) Provisional application No. 60/244,134, filed on Oct. 30, 2000.
(60) Provisional application No. 60/251,332, filed on Dec. 6, 2000.

(51) Int. Cl. C12Q 1/68; G01N 33/53; B05D 3/00; C12M 1/34

(52) U.S. Cl. 435/6; 435/7.1; 435/287.2; 427/2.11; 438/1

(57) ABSTRACT

The present invention describes molecular arrays that can be stacked to generate three-dimensional arrays for detection of molecules of interest in a sample, determination of composition of a complex mixture of molecules, and comparison of composition of two or more samples of molecules, such molecules including DNA, RNA and proteins. Methods to fabricate and use such arrays are also described.

### CROSS-REFERENCE TO RELATED APPLICATIONS

This application claims the priority of U.S. Provisional Application No. 60/244,134, filed Oct. 30, 2000, and U.S. Provisional Application No. 60/251,332, filed Dec. 06, 2000. This application is related to U.S. Non-Provisional Patent Application, entitled “Fluidic Arrays”, filed Oct. 25, 2001, with first named inventor Rajan Kumar, which application is incorporated herein by reference.

STATEMENT REGARDING FEDERALLY SPONSORED RESEARCH OR DEVELOPMENT

Not Applicable

### BACKGROUND OF THE INVENTION

The present invention lies in the field of molecular biology and is particularly concerned with the technique of microarrays used for detection of molecules of interest in a sample, determination of composition of a complex mixture of molecules, and comparison of composition of two or more samples of molecules, such molecules including although not exclusively, DNA, RNA and proteins.

Sequencing of genomes has generated a growing body of sequence information that promises to revolutionize experimental design and data interpretation in pursuit of biological understanding. However, collection of sequence data is not sufficient to decipher the roles of genes and gene products in cellular and organismal function. Therefore, there has been a concomitant growth in development of technologies to exploit the massive amount of DNA sequence data.

One of such revolutionary technologies to emerge in the biotechnology area is the microarray technology. DNA microarrays, consisting of high-density arrangements of oligonucleotides or complementary DNAs (cDNAs) can be used to interrogate complex mixtures of molecules in a parallel and quantitative manner.

The applications of the microarrays are driven by their increasing use in diagnostic testing and genomic research at academic institutions, biotechnology and pharmaceutical companies. In the recent years, the main driver has been genomic analysis.

Microarrays are generated on glass substrates, usually 1 mm thick slides, with a size of 1 inch by 3 inches. The micro arrays are created by depositing molecules of interest on one surface of the glass substrate. The number of molecules on an array is limited by the amount of surface area available.

The microarrays are used to measure the concentrations of nucleic acid populations in a sample by hybridization. Typically, a large number of DNA fragments (called probes) are attached to a solid substrate to create an array. Each probe is attached to a defined place. The nucleic acids in the sample (called targets) are labeled usually by fluorescent dyes, typically fluorescein, Cy3 and/or Cy5. When the array of probes is exposed to the sample, the target nucleic acids in the sample hybridize to specific probes on the array. By shining light of appropriate wavelength, the array is then visualized to determine which probes are hybridized thereby giving an estimate of the nucleic acids present in the sample.

Currently, there are two different technologies used to make microarrays—in situ synthesis method; and Deposition of pre-synthesized DNA.

The two methods differ in the length of the probes deposited. In situ synthesis methods typically use small-length probes due to complexity of individual synthesis steps. For example, the Affymetrix microarrays consist of 20-mer probes. The deposition of presynthesized DNA can involve longer probes, even complete cDNAs (complementary DNAs that are made from reverse transcription of the messenger RNAs present in the cell). Alternatively, the Polymerase Chain Reaction products can be used as probes. The limitations of current technologies include high cost of manufacture, low resolution and sensitivity, lack of customization, low array density, and requirement of specialized and expensive instrumentation.

A method for fabricating microarrays of biological samples has been described (see Brown et. al., U.S. Pat. No. 5,807,522). The method involves dispensing a known volume of reagent at each selected array position, by tapping a capillary dispenser on the support under conditions effective to draw a defined volume of liquid onto support. The method can be used to dispense distinct nucleic acids in discrete spots and therefore, to create microarrays of about 100 or about 1000 spots per 1 square centimeters. Each spot is created by dispensing a volume of liquid between 0.002 and 0.25 nl.

Heyneker (U.S. Pat. No. 6,067,100) teach another method for fabricating arrays of oligonucleotides comprising a solid substrate comprising a plurality of different oligonucleotide pools, each oligonucleotide pool arranged in a distinct linear row to form an immobilized oligonucleotide stripe, wherein the length of each stripe is greater than its width. The oligonucleotides are attached to the solid matrix covalently. Alternatively, each oligonucleotide species is attached to fibers individually and then assembled into a strip on a solid support. Such strips from multiple oligonucleotide pools can be arranged side to side on a solid support to obtain a composite array. The presence of a solid support backing, which preferably is plastic, is always necessary and the use of these arrays in the absence of a solid support is not contemplated.

Walt et al (U.S. Pat. No. 5,244,636) describe a fiber optic sensor which is able to conduct multiple assays and analysis concurrently using molecules immobilized at individual spatial positions on the surface of one of the ends of the optical fiber bundle. The fiber optic bundle can be used to transmit excitation light of suitable wavelength to the molecules at the optical fiber end and also for transmission of the emission light back for detection. An array of oligonucleotides or peptides or any other molecules can be created on the ends of optical fibers and used as a microarray.

A use of protein microarrays has been described by MacBeath et. al. Miniaturized assays were developed that accommodate extremely low sample volumes and enable the rapid, simultaneous processing of thousands of proteins. A high-precision robot was used to spot proteins onto chemically derivatized glass slides at high spatial densities. The proteins attached covalently to the slide surface yet retained their ability to interact specifically with other proteins, or with small molecules, in solution. Three applications for the protein microarrays thus generated were described: screening for protein-protein interactions, identifying the substrates of protein kinases, and identifying the protein targets of small molecules.

Multiple uses of microarrays have been described. One of the primary applications is determination of the nucleic acid or protein composition of a sample. Fodor et al (U.S. Pat. No. 5,800,992) detail a method to compare the composition of two or more samples by labeling members of each of the samples with a distinct labeling molecule, preferably fluorescent molecules. The microarrays described by Fodor et al have at least 1,000 distinct polynucleotides per cm².

It is, therefore, one object of the present invention to provide improved microarrays and novel methods to make the same.

### BRIEF SUMMARY OF THE INVENTION

In general the invention involves the fabrication and use of molecular arrays on fenestrated substrates such arrays comprising distinct zones of molecular depositions on a solid substrate separated by holes. The arrays comprise molecules, usually oligonucleotides or peptides, attached, usually covalently, to the surface of a substrate that has at least one hole or fenestration to allow passage of the target molecules across the substrate. When used in combination with a sample to be analyzed, the holes or fenestrations in the substrate are of sufficient size to allow the target to flow freely across the substrate.

It is another object of the present invention to describe molecular arrays of the invention fabricated by assembly of multiple linear substrates, and methods to make such arrays.

It is another object of the present invention to describe a molecular array created on a substrate with at least one fenestration, comprising the elements of the array being deposited on both surfaces of the said substrate.

One of the advantages of the microarrays of the present invention is that the target molecules in the sample solution are freely mobile across the substrate. Such mobility allows a number of such arrays to be stacked together to fabricate high-density three-dimensional arrays. These three-dimensional arrays comprise a plurality of molecular arrays of the invention such that the target molecules are able to freely move among the layers formed by different surfaces.

It is, therefore, another object of the present invention to describe 3-D microarrays fabricated by stacking multiple 2-dimensional arrays of the invention.

It is another object of the present invention to describe the use of arrays of the invention in molecular analysis of samples.

It is another object of the present invention to describe molecular arrays of the invention, comprising the substrate used for fabrication of the arrays has a thickness less than one hundred microns.

### BRIEF DESCRIPTION OF THE SEVERAL VIEWS OF THE DRAWING

FIG. 1 shows the top view of one embodiment of the array of the invention.

FIG. 2 shows the bottom view of the same array as shown in FIG. 1.

FIG. 3 shows the top view of another embodiment of the array of the invention.

FIG. 4 shows the bottom view of the same array as shown in FIG. 3.

FIG. 5 shows the side view of a composite array comprising six arrays (20, 22, 24, 26, 28, and 30) of the invention joined together by edge pieces.

FIG. 6 shows the top view of the composite array shown in FIG. 5.

FIG. 7 shows ten linear substrates arranged parallel to each other for assembly into an array of the invention as shown in FIG. 8.

FIG. 8 shows top view of the array of the invention comprising assembly of ten linear substrates (shown in FIG. 7) carrying molecular arrays using edge pieces 56 and 58.

FIG. 9 shows the side view of the same array as shown in FIG. 8.

### DETAILED DESCRIPTION OF THE INVENTION

Before providing a detailed description of the inventions of this patent, particular terms used in the patent will be defined.

An “array” is a device comprising a substrate that contains on its surface distinct spots or deposits of one or more than one molecular species. An example of an array in common use is the DNA microarray.

An “element” of an array is a distinct spot or deposition of molecules in a spatially localized area on the substrate of the array.

“Hybridization” is the process by which two strands of DNA or RNA come together to form a double-stranded molecule. For hybridization between two strands to take place, the sequence of the two strands must be completely or nearly so complementary.

“Complementary” strand of a given strand is a strand of DNA or RNA that is able to hybridize to the given strand and is characterized by the presence of nucleotides A, C, G, and T, respectively opposite to nucleotides T, G, C, and A, respectively, on the given strand.

A “Fenestration” in a solid substrate is a continuous channel or hole extending from one surface of the substrate to the opposite surface.

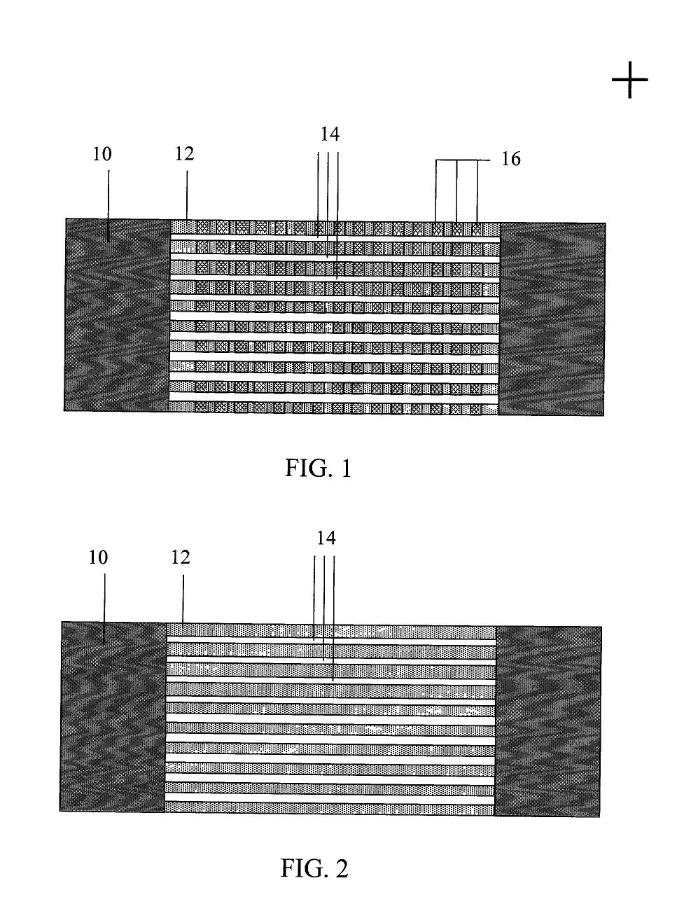

One embodiment of the array of the present invention is described with reference to FIG. 1, which shows the top view of the array. A solid substrate 10 with two planar surfaces provides a support for the molecular array to be generated. The substrate 10 consists of fenestrations 14 that fragment the surface of the substrate into segments 12. Elements of the molecular array 16 are deposited on the segments 12. The elements 16 consist of DNA, RNA, protein or any other chemical or biological species. The size of fenestrations 14 is sufficiently large to allow passage of target molecules, across the substrate, from one side to another. FIG. 2 shows the bottom view of the same array, demonstrating that the molecular depositions are made on only one of the two planar surfaces of the substrate.

A number of materials and methods can be used to fabricate such fenestrated arrays of the invention. The substrates that could be used for fabricating the arrays include, but are not limited to, glass and plastics, such as polystyrene and polycarbonate. If glass is used as the substrate, the fenestrations in the substrate can be produced by using a glass etchant, such as Hydrofluoric Acid (HF). Alternatively, the fenestrations can be produced by laser etching of glass. Fenestrations in plastic substrates can be similarly produced using machining and etching. Both glass and plastic substrates can also be produced by molding. In addition, the array of the invention can be fabricated by first spotting a planar substrate with array elements, and thereafter areas of the substrate in between the spots can be removed, for example by a method described above. The size of the substrate used to create the arrays can be between 5 to 100 millimeters wide and 5 to 100 millimeters long, preferably being 25 millimeters wide and 76 millimeters long, the later being the size of glass slides commonly used for histochemical studies and for conventional DNA array fabrication. The thickness of the substrate can be between 1 microns and 2 millimeters, preferably between 20 microns and 100 microns.

In a preferred embodiment, the substrate will have more than one fenestration, a more preferable embodiment will have between 10 and 100 fenestrations. The smallest dimension of each of the holes is larger than the size of the molecules expected to pass through the holes, typical size being larger than 100 nanometers. The holes can be of any shape with preferred shape being rectangular with the length much larger than the width, with the width being one of the smallest dimensions mentioned above.

Depositions of molecules in array elements can be similarly produced by one of many different methods known to those skilled in the art. A number of microarray spotters are easily available and can be used to spot arrays of molecules on the substrate. The spot sizes are typically 250 microns in diameter; however, spots as small as 75 microns can be deposited with these microarrayers. The volume of liquid used to deposit the probe usually is between 0.2 nanoliters and 100 nanoliters per spot.

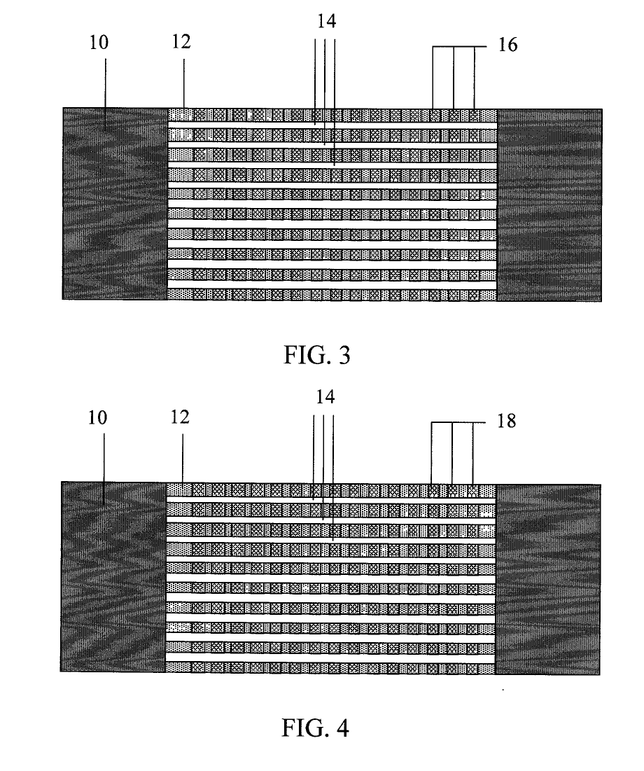

FIG. 3 shows the top view of an alternative embodiment of the invention, which is similar to the top view of the first embodiment shown in FIG. 1. However, FIG. 4 shows the bottom view of the array showing that the bottom surface of the substrate carries array elements 18. The identity of array elements 16 on the top surface and identity of array elements 18 on the bottom surface could be identical or different. If the arrays on the two surfaces consist of identical spots, they can be detected simultaneously or separately. The advantage of simultaneous detection is higher sensitivity; the advantage of having different spots and separate detection is increase in density of array elements.

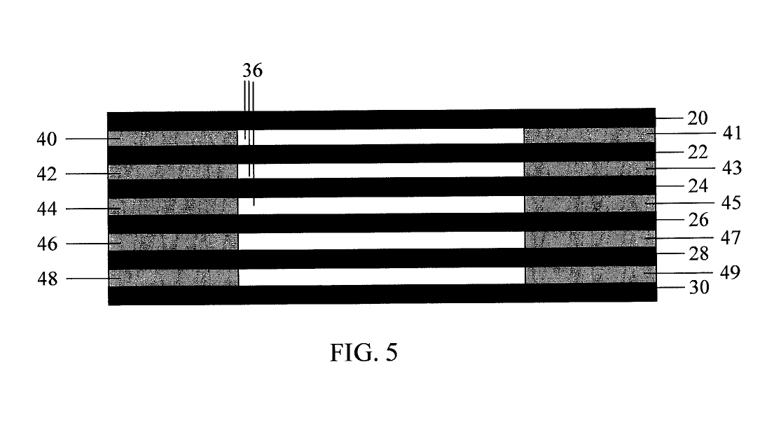

One of the major advantages of the fenestrated two-dimensional arrays of the invention is that the target molecules are able to diffuse rapidly between sample volumes present next to the two surfaces through the fenestrations in the substrate. Because of this ability of rapid diffusion of the target molecules between samples on two sides of the array, by stacking multiple two dimensional arrays, a three-dimensional composite array can be built in which the target molecules have easy access to all array elements in the composite array. An example of such a three-dimensional array embodiment is described with reference to FIG. 5, which shows a side view of the composite array comprising six two-dimensional arrays. Six two-dimensional arrays of the invention are indicated by arrays 20, 22, 24, 26, 28, and 30. The adjacent of the six arrays 20, 22, 24, 26, 28, and 30 are joined together by end pieces 40, 42, 44, 46, and 48 on one end and 41, 43, 45, 47, and 49 on the other end leaving intervening spaces 36 in between the adjacent array. Specifically, for example, array 20 and array 22 are joined together with end pieces 40 and 41, array 22 and array 24 are joined together with end pieces 42 and 43, etc. as shown in FIG. 5. It is important to note that intervening spaces 36 are not identical to spaces 14 in FIG. 1.

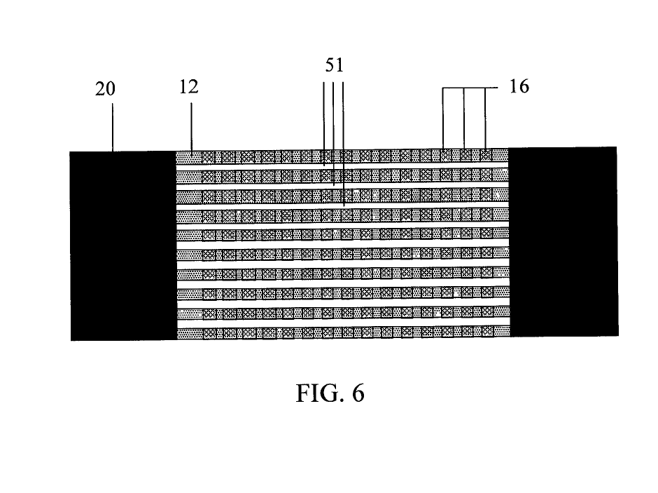

FIG. 6 shows the top view of the same three-dimensional array as shown in FIG. 5. The main point to note is that the spaces 51 between substrate parts 12 are different from intervening spaces 36. However, each of the spaces 51 is fluidically continuous with each of the intervening spaces 36, such that any target molecule present anywhere in the space created by 51 and 36 can traverse to every other point in that space. Only array 20 is visible, other arrays being behind array 20, and therefore, not visible in the top view. The molecular species deposited on array elements 16 of array 20 are preferably different from the molecular species deposited on array elements 16 of array 22, 24, 26, 28, and 30. Two-dimensional arrays fabricated in any of the various techniques can be used to assemble three-dimensional arrays as long as there are fenestrations in the substrates used to assemble the composite array. The joining pieces are not required as long as there is no contact between adjacent two-dimensional arrays. During the stacking, the fenestrations 51 on different arrays 20, 22, 24, 26, 28, and 30 could be in alignment or not in alignment. If the fenestrations 51 in substrates are large enough and do not cover the surfaces of the substrates where the molecules are deposited, the stack could be built without using intervening spaces 36 among different arrays.

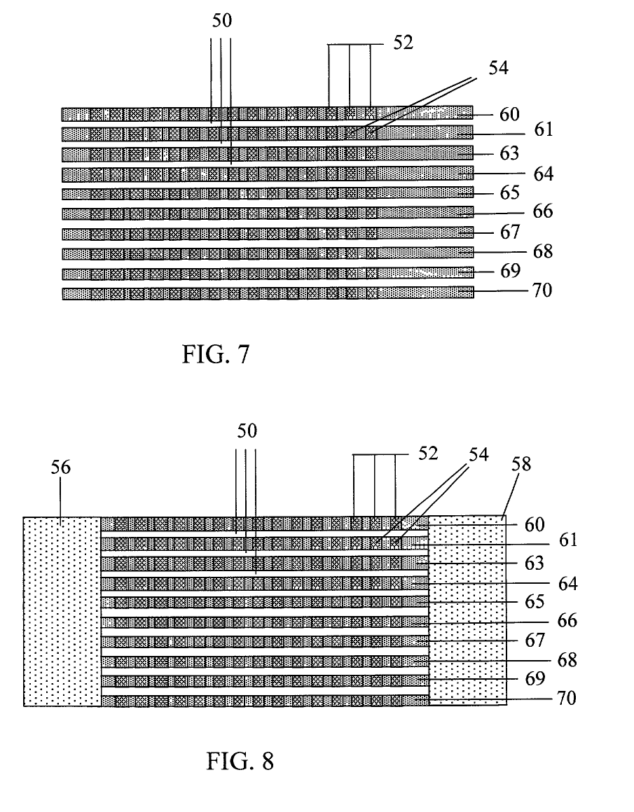

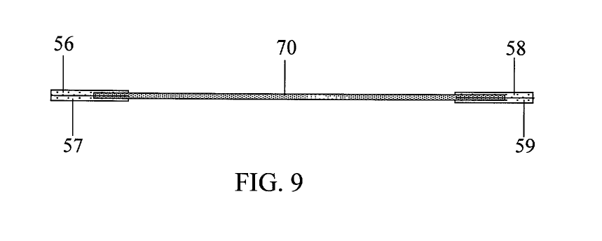

Another method that can be used to create the arrays of the invention is to assemble multiple linear arrays. The method to assemble such two-dimensional arrays is shown in FIG. 7, FIG. 8 and FIG. 9 that comprises of three steps: 1) fabricate multiple linear arrays 60, 61, 62, 63, 64, 65, 66, 67, 68, 69, and 70 comprising of different or similar array elements 52 and 54; 2) arrange them parallel to each other leaving a gap 50 between each adjacent pair of arrays; 3) attach them together solid substrates 56 and 57 on one end and substrates 58 and 59 on the other end, while maintaining them in a parallel configuration. There is no need of a backing matrix. It will be immediately obvious to anyone skilled in the art that the array described in FIG. 4C can be stacked atop each other to create a three-dimensional array that still maintains its ability to be introduced into a fluidic device. Two-dimensional arrays fabricated by this method can also be assembled into three-dimensional arrays. In addition to first fabricating two-dimensional arrays and subsequent arrangement into a three-dimensional array, linear arrays can be arranged in a three-dimensional space to directly create a three-dimensional array.

The methods and materials used to fabricate linear arrays to generate array of the invention are described in detail in the related application, entitled “Fluidic Arrays”, filed Oct. 25, 2001. Specifically, the linear arrays can comprise deposited molecules on one surface of the substrate, both surfaces or preferably all around the circumference of the substrate. The substrate could be square, rectangular or preferably round. Additionally for polymeric biological molecules such as DNA, proteins and RNA, the appropriate molecular deposition can be added to the fiber using in situ synthesis using photolithography or ink jet printing.

The array elements of a two-dimensional array can be investigated for signal associated with each element by a number of methods known to those skilled in the art. A preferable method for detection is using fluorescence labels on the target molecules and detecting the fluorescence signal associated with each element using a fluorescence scanner, a commonly available laboratory tool. Similarly, the array elements of a three-dimensional array can be analyzed for fluorescence of each element in situ using confocal microscope optics, which allows visualization of each individual layer of the three-dimensional array. A review of confocal microscopy is provided by Webb (Theoretical Basis of Confocal Microscopy, Methods in Enzymology, Vol. 307, Pages 3-20. When confocal microscopy is used for detection, there is no need to disassemble the composite array. Alternatively, after the assay, the three-dimensional composite array can be disassembled, for example, into its component two-dimensional arrays, and then each of the individual component two-dimensional array can be analyzed with the conventional scanner.

Another method for analyzing the arrays is to use the substrates used to create the arrays as optical path for excitation light. If the substrate is optically transparent, and optical continuity is maintained between substrate location where the array elements are and the edge of the two-dimensional array, the array elements can be excited by transmitting light through the substrate. In a three-dimensional array, the array elements of each of the two-dimensional arrays can be excited separately with excitation light for imaging the fluorescence associated with the array elements.

### EXAMPLE 1:

### Fabrication of a DNA Array

To fabricate an array as is shown in FIG. 1, take a twenty five millimeters wide, seventy six millimeters long and one hundred micron thick substrate of borosilicate float glass. Using a syringe, place a glass etchant solution on the substrate in nine lines 500 micron apart. Allow the glass etchant to etch through the glass. After rinsing the glass substrate with water, attach amino functional groups to the surface of the substrate by treating it with N-(2-Aminoethyl)-3-aminopropyltrimethoxysilane. Spot human cDNA molecules of interest on the substrate using a standard microarrayer. Allow the cDNA molecules to attach to the amino groups and wash. Dry the substrates. The arrays are now ready for use.

### EXAMPLE 2

### Fabrication of a Three-Dimensional Composite DNA Array

To fabricate an array shown in FIG. 5, take six substrates similar to the substrate used in example 1. Using the methods described in example 1, create fenestrations in the substrate and then deposit molecular arrays on each of the six substrates, taking care to leave an area of the substrate extending 15 millimeters from each end unetched and not deposited with molecular elements. Take ten pieces of polycarbonate 10 millimeters wide, 25 millimeters long and 0.5 millimeters thick and use them as end pieces 40-49. Stack the six arrays atop each other using two end pieces in between two arrays and sticking them together with a strong adhesive, such as super glue. The three-dimensional array is ready for use.

### EXAMPLE 3

### Use of an Array for Analysis of a cDNA Sample

Make an array as described in example 1 using human cDNA. Place the array in a chamber slightly larger than the array and with a volume of 2 ml. Take an RNA sample from the tissue of interest and prepare cDNA using a reverse transcriptase reaction. Label the cDNA molecules present in the sample with Cy3. Add the fluorescently labeled sample and introduce it into the chamber containing the array. Let the target molecules in the sample hybridize to the probes for 1 hour. Take the array out and wash with 0.1 mM TE buffer (10 mM Tris HCl, 0.5 mM EDTA). Position the array under a fluorescent microscope equipped with a digital camera. Use an excitation light of 550 nm wavelength and observe and record the light intensity from each element at 570 nm emission wavelength. If the sample contains target molecules that are complementary to the probes on the array elements, the light intensity recorded from the corresponding element(s) will be stronger than others.

### EXAMPLE 4

### Analysis of The Array Elements of a Composite Three-Dimensional Array by Confocal Microscopy

Fabricate a three-dimensional array as in example 2. Expose the array to targets in a sample and let the hybridization take place. After hybridization and a rinse as in example 3, place the array on a confocal fluorescence microscope and record the light intensity associated with each array element using excitation and emission filters as in example 3 as follows. First, bring the array elements on the array 20 into focus. Record the intensity of light emitted. Move the array appropriately to bring each array element into field of view and record corresponding light intensity. After all the array elements on array 20 have been investigated, bring array elements of array 22 into focus. Repeat the process and record light intensity for each array element. Continue the process until all array elements on all arrays comprising the composite three-dimensional array have been investigated. If the sample contains target molecules that are complementary to the probes on any array elements, the light intensity recorded from the corresponding element(s) will be stronger than others.

Although the invention has been described in some detail by way of illustration and example for purposes of clarity and understanding, it may be readily apparent to those of ordinary skill in the art in light of the teachings of this invention that certain changes and modifications may be made without departing from the spirit or scope of the appended claims.

What I claim as my invention is

1. Any array of molecules created on surface of a substrate comprising the said substrate contains fenestrations larger than 1 micrometer in dimensions.

2. An array of claim 1, comprising of array elements, the said array elements deposited on the surface of the substrate, the surface of the substrate being made discontinuous by the fenestrations in the substrate.

3. An array of claim 1, comprising a substrate that contains more than 1, but less than 100 fenestrations.

4. An array of claim 1, comprising a substrate that contains 100 or more fenestrations.

5. Any method to fabricate an array of claim 1.

6. A method to fabricate an array of claim 1, comprising
a. Deposition of array elements on the substrate, and
b. Removal of substrate between array elements to create fenestrations.

7. A method to fabricate an array of claim 1, comprising
a. Removal of substrate at defined locations to create fenestrations,
b. Deposition of array elements on the substrate between the fenestrations.

8. A method to fabricate an array of claim 1, comprising
a. Creating rows of array elements on linear substrates, and
b. Joining together said substrates at both edges.

9. An array of claim 1 comprising array elements composed of DNA, RNA or proteins or a surface modification.

10. Any array of claim 1 comprising a substrate with less than 0.5 mm thickness.

11. Any composite array of molecules comprising two or more of arrays of claim 1 stacked together.

12. A composite array of claim 11, comprising different arrays of claim 1 are not in contact with each other.

13. A composite array of claim 11, in which the at least one of the array elements present on each of the arrays of claim 1 is not present on any other arrays of claim 1.

14. The use of an array of claim 1 for detection of analytes present in a sample.

15. Claim 14 where the analytes to be detected are DNA, RNA or proteins.

16. Any array of molecules on surface of a substrate comprising the said substrate has a thickness of less than 0.5 mm.

17. Any array of claim 16, comprising a substrate with less than 100 micron thickness.

18. Any array of claim 16, comprising the substrate is glass.

19. Any method of sample analysis using a composite array of claim 11.

20. Any method of sample analysis using a composite array of claim 11 comprising the array elements are imaged using a confocal optical device.
